(19) United States
(12) Patent Application Publication
(10) Pub. No.: US 2026/0345052 A1
(43) Pub. Date: Nov. 5, 2026

(54) BIOLOGICAL INFORMATION MEASUREMENT DEVICE

(71) Applicant: OMRON HEALTHCARE Co., Ltd., Kyoto (JP)

(72) Inventors: Shuhei MUNEHIRA, Kyoto (JP); Moe KATO, Kyoto (JP)

(21) Appl. No.: 19/713,991

(22) Filed: Jun. 19, 2026

Related U.S. Application Data
(63) Continuation of application No. PCT/JP2025/000788, filed on Jan. 14, 2025.

(30) Foreign Application Priority Data
Mar. 14, 2024 (JP) 2024-040362

(51) Int. Cl. A61B 5/318; A61B 5/00; A61B 5/339

(52) U.S. Cl. CPC A61B 5/318; A61B 5/339; A61B 5/681

(57) ABSTRACT

A biological information measurement device including at least a first electrode and a second electrode, and configured to measure an electrocardiographic waveform based on a potential difference between the first and second electrode, the biological information measurement device including a main body case including a side surface formed of a conductive material and functioning as the first electrode, a bottom surface on which the second electrode is disposed and which abuts against the human body when being attached, and a top surface facing the bottom surface, an electrocardiographic signal detection circuit that detects signals related to potentials of the first and second electrode, a first electrical path that electrically connects the first electrode to the electrocardiographic signal detection circuit, and a second electrical path on which an electrostatic discharge protection element is mounted and that connects the first electrode to the second electrode via the electrostatic discharge protection element.

### CROSS-REFERENCE TO RELATED APPLICATIONS

This application is the U.S. National stage application filed pursuant to 35 U.S.C. 365(c) and 120 as a continuation of International Patent Application No. PCT/JP 2025/000788, filed Jan. 14, 2025, which application claims priority to Japanese Patent Application No. 2024-040362, filed Mar. 14, 2024, which applications are incorporated herein by reference in their entireties.

### TECHNICAL FIELD

The present invention belongs to the technical field related to healthcare, and particularly relates to a biological information measurement device.

### BACKGROUND ART

It has recently become common for an individual to measure information (hereinafter, also referred to as biological information) related to a body and health of the individual such as a blood pressure value and an electrocardiographic waveform on a daily basis by himself/herself by using a measurement device and utilize the measurement result for health management. Accordingly, there has been an increasing demand for apparatuses with an emphasis on portability, and many portable measuring devices have been proposed (for example, Patent Document 1 and the like).

Patent Document 1 discloses a wristwatch-type biological information measurement device that includes an electrocardiographic electrode and that can measure an electrocardiographic waveform. In a small-sized device such as the wristwatch-type wearable device, since the capacitance of the GND is not sufficient, in a case where a screen display unit (display) is disposed downward in an electrostatic discharge (ESD) test or the like, when a case is a metal, static electricity enters a control board connected to the ground (GND) of the device from the metal case to which ESD is applied, and thus a malfunction of the control board or destruction of components may occur.

As a technique for preventing the malfunction of the control board and a damage to electronic circuit components mounted on the control board due to the static electricity that has entered the metal case, it is known to set the metal case to GND (connect the metal case to GND), although this is a technical field different from that of the electrocardiograph (for example, Patent Document 2). In the technique described in Patent Document 2, the electrostatic resistance is improved by electrically connecting the conductive aluminum case to the ground of the control board.

### CITATION LIST

### Patent Literature

Patent Document 1: JP 2024-14478 A
Patent Document 2: JP 2014-181562 A

### SUMMARY OF INVENTION

### Technical Problem

However, when a case or the like that can be touched by a human body is grounded in a device such as an electrocardiograph, the device cannot pass a withstand voltage test or a leakage current test from the viewpoint of safety standards (because a current flows between the ground, which is a metal case, and an electrode for electrocardiographic measurement when a voltage is applied).

In view of the above-described problems, an object of the present invention is to provide a technique for obtaining a high electrostatic resistance in a biological information measurement device including a metal case and provided with an electrode.

### Solution to Problem

A biological information measurement device according to the present invention adopts the following configurations in order to solve the above problems. Specifically, provided is a biological information measurement device used by being attached to a human body, including at least a first electrode and a second electrode, and configured to measure an electrocardiographic waveform based on a potential difference between the first electrode and the second electrode, the biological information measurement device including
a main body case including a side surface formed of a conductive material and functioning as the first electrode, a bottom surface on which the second electrode is disposed and which abuts against the human body when being attached, and a top surface facing the bottom surface,
an electrocardiographic signal detection circuit that detects signals related to potentials of the first electrode and the second electrode,
a first electrical path that electrically connects the first electrode to the electrocardiographic signal detection circuit, and
a second electrical path on which an electrostatic discharge protection element is mounted and that connects the first electrode to the second electrode via the electrostatic discharge protection element.

That is, the first electrode and the second electrode are electrically connected to each other via the electrostatic discharge protection element only when a current exceeding a specific frequency and a specific applied voltage is generated due to an electrostatic discharge phenomenon or the like. With such a configuration, even when the static electricity enters the biological information measurement device via the second electrode or the like, the static electricity can be released to the main body case serving as the first electrode via the electrostatic discharge protection element provided on the signal line for electrocardiographic measurement through which a current easily flows.

Thus, it is possible to provide the high electrostatic resistance even to a small-sized biological information measurement device such as a wristwatch-type in which a large ground cannot be provided. Further, since the main body case to which the static electricity is released is the electrode for electrocardiographic measurement, it is not necessary to apply a high voltage in the withstand voltage test or the leakage current test of the device, and there is no problem in passing the withstand voltage test. Note that, as a path for releasing the static electricity, the static electricity can be further released to the earth outside the device via the main body case serving as the first electrode, but the present invention is intended to release the static electricity to the metal case of the biological information measurement device.

The electrocardiographic signal detection circuit is provided on a first substrate disposed in the main body case in a direction parallel to the bottom surface, and a part of the first electrical path and a part of the second electrical path may be provided on a second substrate that is disposed in the main body case in a direction orthogonal to the bottom surface and is joined to the first electrode by a conductive induction tape to be electrically connected to the first electrode.

With this configuration, a signal line (that is, a route through which the static electricity flows toward the case) that efficiently connects the first electrode to the second electrode can be constructed in the main body case having a small space.

The first substrate and the second substrate may be integrally formed as a rigid flexible substrate. With such a configuration, the electrocardiographic signal detection circuit (first substrate portion) and a portion (second substrate portion) electrically connected to the first electrode can be disposed in an L shape in the case, and the number of components constituting the device can be reduced.

A display is provided on the top surface, and the first electrode and the display may be joined to each other by a conductive tape to be electrically connected to each other. With such a configuration, even when ESD occurs when the display is directed downward in an ESD test or the like, the static electricity easily flows from the side surface of the main body case to the display that is the top surface of the main body case, and it is possible to more suitably prevent the static electricity from flowing to the electronic components in the main body case.

The biological information measurement device may include a third substrate disposed in a direction parallel to the first substrate in the main body case and on which a control device that controls the biological information measurement device is mounted, an operation button disposed so as to protrude from the side surface, and a fourth substrate provided with a part of an electrocardiographic signal line that connects the electrocardiographic signal detection circuit to the control device and disposed in a direction orthogonal to the first substrate at a position different from the second substrate. With such a configuration, the space in the main body case can be more efficiently utilized.

Note that the configurations and processing described above can be combined with one another to constitute the present invention unless the combination leads to technical contradiction.

### Advantageous Effects of Invention

According to the present invention, a technique for obtaining a high electrostatic resistance in a biological information measurement device including a metal case and provided with electrode can be provided.

### BRIEF DESCRIPTION OF DRAWINGS

FIG. 1 is an external perspective view illustrating an outline of a biological information measurement device according to an embodiment of the present invention.

FIG. 2 is a side view illustrating the outline of the biological information measurement device according to the embodiment.

FIG. 3 is an external view when a main body portion of the biological information measurement device according to the embodiment is viewed from a bottom portion side.

FIG. 4 is a schematic cross-sectional view when the biological information measurement device according to the embodiment is viewed from a side.

FIG. 5 is a schematic cross-sectional view of the vicinity of a sensor substrate housing portion of the biological information measurement device according to the embodiment.

FIG. 6 is a schematic cross-sectional view for describing connection between electrodes and a sensor substrate and a bezel conductive substrate of the biological information measurement device according to the embodiment.

FIG. 7 is a schematic circuit diagram illustrating a signal line between an electrode and the electrocardiographic signal detection circuit of the biological information measurement device according to the embodiment.

FIG. 8 is a block diagram illustrating a functional configuration of the biological information measurement device according to the embodiment.

### DESCRIPTION OF EMBODIMENTS

### Embodiments

Embodiments of the present invention will be specifically described below with reference to the drawings. Note that unless otherwise specified, the dimensions, materials, shapes, relative arrangements, and the like of the configurations described in the following embodiments are not intended to limit the scope of the present invention to those alone.

### Overall Configuration of Device

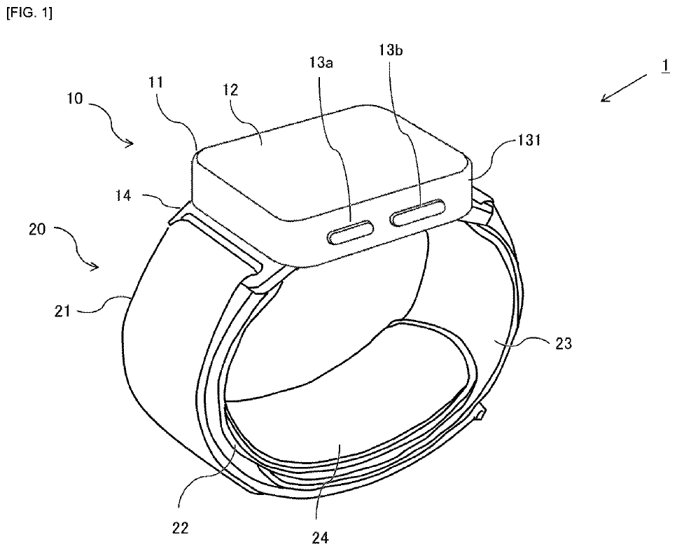

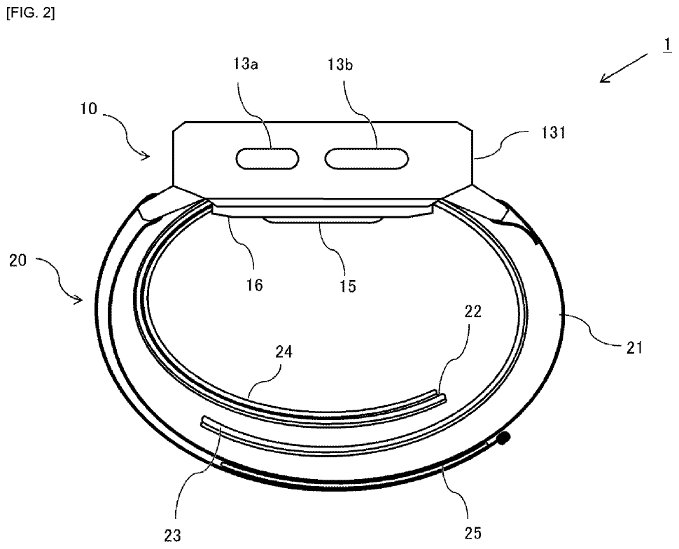

FIG. 1 is an external perspective view illustrating an outline of a configuration of a biological information measurement device 1 according to the present embodiment. In addition, FIG. 2 is a side view illustrating the outline of the configuration of the biological information measurement device 1 according to the present embodiment. As illustrated in FIG. 1 and FIG. 2, the biological information measurement device 1 is generally a wristwatch-type wearable device including a main body portion 10 and a belt portion 20, and is capable of measuring biological information such as an electrocardiographic waveform, a pulse wave (pulse), and a blood pressure value while being attached on a wrist of a human body.

As illustrated in FIG. 1 and FIG. 2, the main body portion 10 includes a main body case 11 and a cuff cover 16 described below. The main body case 11 is provided with a display 12 (for example, an organic EL display or the like), a bezel 131, operation buttons 13a and 13b, a lug 14, and the like, and is also provided with a sensor substrate housing portion 15 in which a sensor substrate is housed. Note that, in the present embodiment, a side on which the display 12 is formed is referred to as a front surface of the main body case 11, and a side on which the sensor substrate housing portion 15 is formed is referred to as a bottom portion of the main body case 11. In addition, in the following description, the front surface side of the main body case 11 may be referred to as the upper side, and the bottom portion side of the main body case 11 may be referred to as the lower side.

The bezel 131 constituting the side surface of the main body case 11 is formed of a conductor (for example, stainless steel), and functions as an electrode (right hand electrode) for electrocardiographic waveform measurement. Therefore, hereinafter, the bezel 131 is also referred to as a first electrode or the like when the bezel 131 is expressed in terms of the function related to the electrocardiographic waveform measurement.

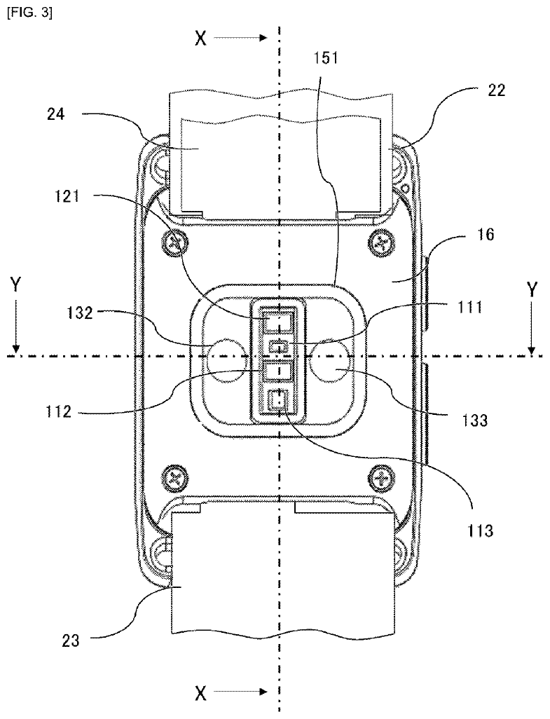

FIG. 3 illustrates an external view of the main body portion 10 when viewed from the bottom portion side. As illustrated in FIG. 3, the bottom portion of the main body case 11 includes a central region covered with a resin cover 151, and a region that corresponds to an outer periphery of the central region and is covered with the cuff cover 16. At least a portion of the resin cover 151 is formed of a light-transmissive resin, and an inner side of the main body case 11 in the region covered with the resin cover 151 corresponds to the sensor substrate housing portion 15. The sensor substrate housing portion 15 is located in the central region of the main body case 11 covered with the resin cover 151 in a plan view, and is formed so as to project toward the wrist with respect to the cuff cover 16 in the attached state, as illustrated in FIG. 2. That is, a surface of the resin cover 151 on the bottom portion side serves as a contact surface that comes into contact with a human body.

In addition, a second electrode 132 and a third electrode 133 are provided at the bottom portion of the main body case 11 such that contact surfaces with the human body are exposed. The second electrode 132 functions as a left hand electrode, and the third electrode 133 functions as a GND electrode (electrode that provides a reference potential for the electrocardiographic waveform measurement). During the electrocardiographic waveform measurement, the biological information measurement device 1 is attached, the contact surfaces of the second electrode 132 and the third electrode 133 are brought into contact with a skin surface at an attachment portion, and the bezel 131 is touched with a finger on a side where the biological information measurement device 1 is not attached. Thus, the electrocardiographic waveform measurement can be performed by I induction. Note that the detailed structures of the second electrode 132 and the third electrode 133 will be described below.

In addition, although not illustrated, a charging terminal is also provided at the bottom portion of the main body case 11. A rechargeable battery (not illustrated in FIG. 3) can be charged by connecting a connection terminal of a power supply device and the charging terminal.

In addition, as illustrated in FIG. 3, a first LED 111, a second LED 113, a first photodiode (PD) 112, and a second PD 121 mounted on a lower surface (mounting surface) of a second sensor substrate 102 described below can be seen through the light-transmissive portion of the resin cover 151 from the bottom portion side of the main body case 11.

The belt portion 20 includes a belt 21 and a surface fastener 25 for fixing the biological information measurement device 1 to the wrist, and also includes a first pressing cuff 22 and a second pressing cuff 23 for compressing an artery in the wrist, and a sensing cuff 24 for detecting a pressure pulse wave. Note that connection portions between the respective cuffs 22, 23, and 24 and the main body case 11 are covered with the cuff cover 16. The cuff cover 16 protects the connection portions between the respective cuffs 22, 23, and 24 and the main body case 11, and also has a function to fix the respective cuffs 22, 23, and 24 to the main body case 11.

### Internal Structure of Case

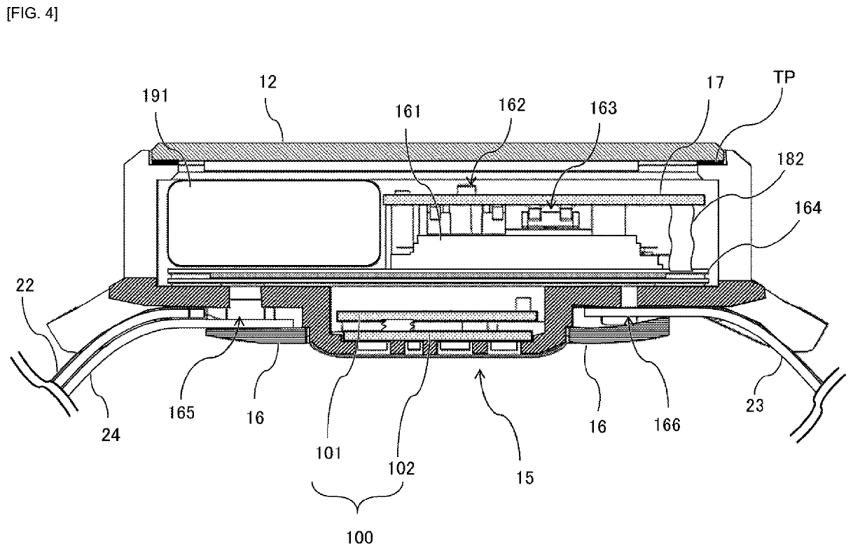

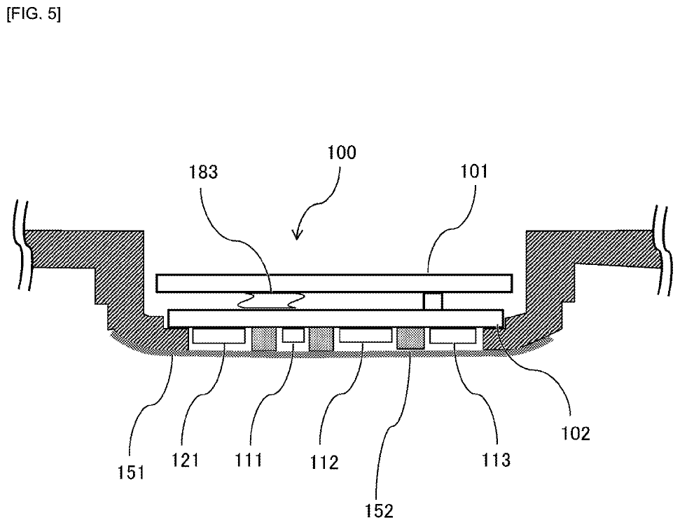

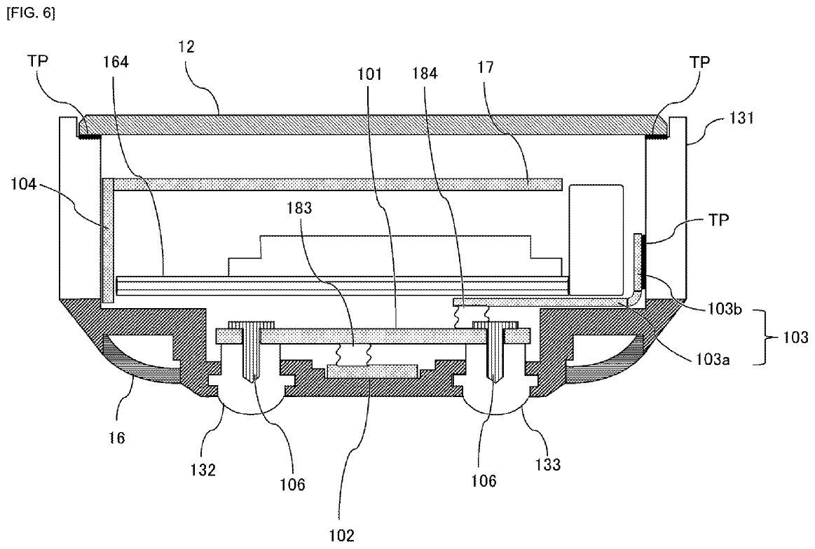

Next, the internal configuration of the main body case 11 will be described with reference to FIG. 4 to FIG. 6. FIG. 4 is a schematic cross-sectional view corresponding to an X-X cross-section of FIG. 3, and FIG. 5 is an enlarged view of the vicinity of the sensor substrate housing portion 15 in FIG. 4. FIG. 6 is a schematic cross-sectional view corresponding to a Y-Y cross-section of FIG. 3. Note that FIG. 4 to FIG. 6 are not accurate cross-sectional views, and the configuration is appropriately omitted and deformed for convenience of description.

As illustrated in FIG. 4, a rechargeable battery 191, a control board 17, a piezoelectric pump 161, a valve 162, a pressure sensor 163, a flow path plate 164, and the like are housed inside the main body case 11. In addition, the sensor substrate housing portion 15 formed in a protrusion shape is provided in the vicinity of the bottom portion of the main body case 11, and a sensor substrate set 100 including a first sensor substrate 101 and a second sensor substrate 102 is housed in the sensor substrate housing portion 15. The bezel 131 constituting the side surface of the main body case 11 is joined to the display 12 constituting the top surface of the main body case 11 with a conductive tape TP.

Further, the bottom portion of the main body case 11 is provided with a first connection portion 165 and a second connection portion 166 in a part of a region where the sensor substrate housing portion 15 is not provided in a plan view. The first connection portion 165 connects the main body case 11 (more specifically, the flow path plate 164 in the case) and the first pressing cuff 22 and the sensing cuff 24. The second connection portion 166 similarly connects the main body case 11 and the second pressing cuff 23. The first connection portion 165 and the second connection portion 166 are covered with the cuff cover 16 provided in the region corresponding to an outer periphery of the sensor substrate housing portion 15 on the bottom portion of the main body. In addition, as described above, the portion where the sensor substrate housing portion 15 is located is covered with the resin cover 151.

As the rechargeable battery 191, a general-purpose rechargeable battery such as a lithium ion battery can be adopted, and the rechargeable battery 191 can be repeatedly charged by receiving power supply via the charging terminal. In addition, the piezoelectric pump 161, the valve 162, the pressure sensor 163, the flow path plate 164, the first pressing cuff 22, the second pressing cuff 23, and the sensing cuff 24 are configurations related to blood pressure measurement.

The flow path plate 164 is a conductive member (metal), and a flow path for feeding gas from the piezoelectric pump 161 to each cuff is formed therein. The flow path plate 164 is electrically connected to the control board 17 by a spring connector 182, and functions as a GND (hereinafter, also referred to as a device GND) of the entire device. In addition, for the first sensor substrate 101, the flow path plate 164 also functions as a shield against noise generated from the internal devices such as the piezoelectric pump 161.

On the control board 17, a processor such as a central processing unit (CPU) not illustrated in the drawing, a memory such as a random access memory (RAM) and the like are mounted to control the entire biological information measurement device 1. As described above, the control board 17 is connected to the flow path plate 164 (device GND), but it is generally difficult to sufficiently secure a GND area in a small-sized device such as a wristwatch-type, and in a case where the static electricity enters the main body case 11 due to the ESD, the static electricity flows to the control board 17, and malfunction or damage of the electronic components on a circuit may occur. In this regard, the biological information measurement device 1 according to the present embodiment is provided with a path for releasing the static electricity to the bezel 131 instead of the device GND, thereby suppressing the flow of the static electricity to the control board 17 and significantly improving the electrostatic resistance. Details will be described later.

### Sensor Substrate Set

Next, the sensor substrate housing portion 15 and the sensor substrate set 100 will be described. As illustrated in FIG. 5, the sensor substrate housing portion 15 is a space projecting from the bottom portion of the main body case 11 toward a side that comes into contact with the human body. The sensor substrate set 100, in which the first sensor substrate 101 and the second sensor substrate 102 are vertically stacked in two stages, is housed in the space. Note that the first sensor substrate 101 and the second sensor substrate 102 are connected by a conductive spring connector 183, and these two substrates function as a pair.

On a lower surface of the second sensor substrate 102, two light emitting elements of the first LED 111 and the second LED 113, and two light receiving elements of the first photodiode (PD) 112 and the second PD 121 are provided. Note that, in the present embodiment, the first LED 111 irradiates green irradiation light, and the second LED 113 irradiates red light and/or infrared light in addition to green light. Isolation walls 152 are also provided so as to isolate the first LED 111, the second LED 113, the first PD 112, and the second PD 121 from each other.

On the other hand, the first sensor substrate 101 is provided with an amplifier circuit that amplifies a biological signal acquired by each sensor, a circuit that performs analog-to-digital (A/D) conversion, and the like. Of course, an electrocardiographic signal detection circuit 50 for measuring the electrocardiographic waveform by a signal detected via the bezel 131, the second electrode 132, and the third electrode 133 is also mounted, and details thereof will be described below.

In this way, the sensor substrate set 100 has a vertically stacked two-stage structure including the second sensor substrate 102 and the first sensor substrate 101. Thus, compared to a case where all components are mounted on one substrate, it is possible to significantly reduce the area of the substrates in a plan view. Note that the first sensor substrate 101 may be a double-sided mounting substrate.

### Configuration of Second Electrode and Third Electrode

As illustrated in FIG. 6, the second electrode 132 and the third electrode 133 are fixed while being in contact with the lower surface of the first sensor substrate 101. In addition, both the electrodes are arranged such that a portion of each of the electrodes projects from a contact surface TS (a surface located on a line indicated by a broken line in FIG. 6), which is a surface on the bottom portion side of the resin cover 151, toward a side that comes into contact with the human body when the device is attached.

Opening portions (not illustrated) is provided in the first sensor substrate 101, and an electrode pad (not illustrated) is formed on an outer periphery of each opening portion. As illustrated in FIG. 6, the second electrode 132 and the third electrode 133 are screwed with screw members 106 serving as male screws via the opening portions of the first sensor substrate 101, and are thereby fixed to the first sensor substrate 101. The fixing is performed in a state in which the distal end surfaces of the second electrode 132 and the third electrode 133 are in contact with the electrode pads formed at the outer peripheries of the opening portions of the first sensor substrate 101. Thus, the second electrode 132 and the third electrode 133 are fixed to the first sensor substrate 101 while being electrically connected to the first sensor substrate 101.

### Substrate Adjacent to Bezel Inner Wall

Further, as illustrated in FIG. 6, a bezel conductive substrate 103 connected to the first sensor substrate 101 by a spring connector 184 and joined to an inner wall of the bezel 131 as the first electrode by the conductive tape TP is disposed in the main body case 11. The bezel conductive substrate 103 is bent in a substantially L shape, and includes a horizontal portion 103a disposed in a direction parallel to the first sensor substrate 101 (that is, a direction parallel to the bottom surface of the main body case) and a vertical portion 103b disposed in a direction orthogonal to the first sensor substrate 101 (along the inner wall of the bezel 131). The terms “parallel, orthogonal, horizontal, and vertical” herein are used for convenience to indicate approximate orientations, and it is not necessary that each portion is actually horizontal or vertical. All or a part of the bezel conductive substrate 103 is formed of a flexible substrate, and thus the bezel conductive substrate 103 can be disposed while being bent.

An operation unit substrate 104 for the operation button 13 is disposed along the inner wall of the bezel 131 facing the vertical portion 103b of the bezel conductive substrate 103 in the main body case 11. The biological signals detected, amplified, and A/D converted by the first sensor substrate 101 are transmitted to the control board 17 via the operation unit substrate 104.

### Electrocardiographic Signal Detection Line

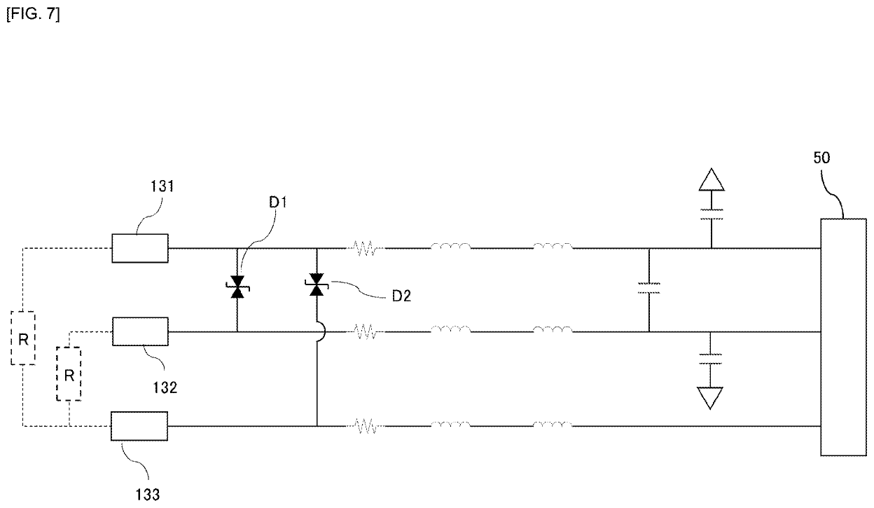

In the case of measuring the electrocardiographic waveform, when the user touches the bezel 131 with the right hand while the biological information measurement device 1 being attached on the left wrist, a signal is sent to the electrocardiographic signal detection circuit 50 of the first sensor substrate 101 via the bezel conductive substrate 103. Here, FIG. 7 schematically illustrates signal lines between the bezel 131, the second electrode 132, and the third electrode 133, and the electrocardiographic signal detection circuit 50. As illustrated in FIG. 7, the second electrode 132 and the third electrode 133 are connected to the bezel 131 via a TVS diode D1 and a TVS diode D2, respectively. The TVS diode D1 and the TVS diode D2 may be disposed on the first sensor substrate 101 or may be disposed on the bezel conductive substrate 103. In FIG. 7, an electrical path connecting the bezel 131 to the electrocardiographic signal detection circuit 50 corresponds to the first electrical path of the present invention, and an electrical path in which the TVS diode D1 is disposed corresponds to the second electrical path of the present invention.

When the ESD occurs in the second electrode 132 and the third electrode 133, unless the second electrode 132 and the third electrode 133 are connected to the bezel 131 via the TVS diode D1 and the TVS diode D2, respectively, the static electricity flows into the control board 17 via the electrocardiographic signal detection circuit 50 and further via the signal line on the operation unit substrate 104. In this regard, as illustrated in FIG. 7, by providing lines connecting the second electrode 132 and the third electrode 133 to the bezel 131 via the TVS diode D1 and the TVS diode D2, respectively, (most of) the static electricity flows to the bezel 131 even when the ESD occurs in the second electrode 132 and the third electrode 133. However, the bezel 131 is the first electrode for the electrocardiographic measurement and is not the GND of the device.

### Functional Configuration of Device

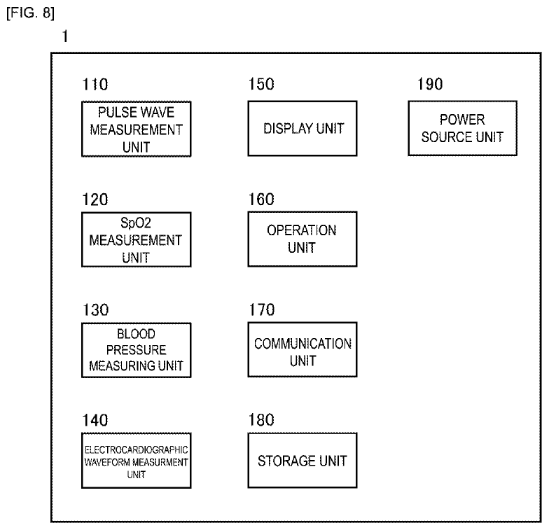

Next, a functional configuration of the biological information measurement device 1 will be described. FIG. 8 is a block diagram illustrating the functional configuration of the biological information measurement device 1. As illustrated in FIG. 8, the biological information measurement device 1 according to the present embodiment includes functional units of a pulse wave measurement unit 110, a blood oxygen saturation (SpO2) measurement unit 120, a blood pressure measurement unit 130, an electrocardiographic waveform measurement unit 140, a display unit 150, an operation unit 160, a communication unit 170, a storage unit 180, and a power source unit 190. The processor of the control board 17 reads a program from the memory and executes the program to control each configuration of the biological information measurement device 1, thereby implementing these functional units.

The pulse wave measuring unit 110 includes the first LED 111, the second LED 113, and the first PD 112, and measures a pulse wave by a so-called photoplethysmographic method to calculate a pulse. Specifically, green light is emitted from the first LED 111 and the second LED 113, and reflected light reflected in a living body is received by the first PD 112, so that a blood flow volume that changes with a pulsation of a heart (a change in volume of a blood vessel) is detected, and a pulse wave is measured.

The SpO2 measuring unit 120 includes the second LED 113 and the second PD 121, and the second PD 121 receives reflection light of red light or infrared light emitted from the second LED 113, so that the SpO2 measuring unit 120 measures blood oxygen saturation from the intensity of the reflection light.

The blood pressure measurement unit 130 includes the piezoelectric pump 161, the valve 162, the pressure sensor 163, the flow path plate 164, the first pressing cuff 22, the second pressing cuff 23, and the sensing cuff 24, and measures a blood pressure by a so-called oscillometric method. The blood pressure measurement using the oscillometric method is a known technique, and therefore the detailed description thereof is omitted.

The electrocardiographic waveform measurement unit 140 includes the bezel 131, the second electrode 132 and the third electrode 133 provided at the bottom portion of the main body case 11, and the electrocardiographic signal detection circuit 50, and measures an electrocardiographic waveform by a so-called I-induction method. Specifically, an electrocardiographic waveform is measured based on a potential difference between the second electrode 132 and the third electrode 133 that are in contact with the wrist of one arm in the attachment state, and a finger of another hand in contact with the bezel 131 functioning as the first electrode.

The display unit 150 includes the display 12, and displays various types of information such as a measurement result of the biological information and a menu screen. The operation unit 160 includes the operation buttons 13a and 13b, and receives inputting operations of the user through them. The communication unit 170 includes an antenna for wireless communication (not illustrated), and performs information communication with another electronic device such as an information processing terminal by, for example, BLE communication. Note that a terminal for wired communication may be provided.

The storage unit 180 includes a main storage device (not illustrated) such as a RAM, and stores various information such as an application program and measured biological information. In addition to the RAM, for example, an auxiliary storage device such as a flash memory may be provided. The power source unit 190 includes the rechargeable battery 191 and the charging terminal, and functions as a power supply source to each unit constituting the biological information measurement device 1.

### Effects of Present Embodiment

As described above, in the biological information measurement device according to the present embodiment, the bezel 131 of the main body case 11 functions as the first electrode, and is connected to the second electrode 132 and the third electrode 133 by the signal lines for the electrocardiographic measurement provided on the bezel conductive substrate 103 and the first sensor substrate 101. The TVS diode D1 and the TVS diode D2 serving as the electrostatic discharge protection elements are mounted on the signal line for electrocardiographic measurement. Accordingly, even when the ESD occurs in the biological information measurement device 1 via the second electrode 132, the third electrode 133, and the like, the static electricity can be released to the bezel 131 via the TVS diode D1 and the TVS diode D2 provided on the signal line for electrocardiographic measurement, through which a current flows more easily than toward the device GND. Further, it is also possible to release the static electricity to the ground outside the device through the display 12 connected to the bezel 131 by the conductive tape TP.

According to this, it is possible to provide the high electrostatic resistance even to a small biological information measurement device such as a wristwatch-type in which a large device GND cannot be provided. Further, since the bezel 131 to which the static electricity is released is the electrode for electrocardiographic measurement, it is not necessary to apply a high voltage in the withstand voltage test of the device, and there is no problem in passing the withstand voltage test or the leakage current test.

### Others

The descriptions of the above examples are merely illustrative of the present invention, and the present invention is not limited to the specific embodiments described above. Various modified examples and combinations may be made within the scope of the technical idea of the present invention. For example, the biological information measurement device is only required to include an electrode and a circuit for measuring an electrocardiographic waveform, and a function and a configuration for acquiring other biological information are not necessarily indispensable.

In the above embodiment, the TVS diode is illustrated as the electrostatic discharge protection element, but other electrostatic discharge protection elements can be adopted. The shapes, holding methods, and arrangement positions of the second electrode 132 and the third electrode 133 are not limited to the examples of the above-described embodiments, and desired shapes, holding methods, and arrangement positions can be adopted.

In the above embodiment, almost the entirety of the device on an upper surface side is the display 12, and the bezel 131 portion functions as the first electrode. However, the upper surface side of the main body case 11 may also be formed of a metal without providing the display on the upper surface side of the device (in this case, the upper surface side also functions as the first electrode).

In the embodiment described above, the bezel conductive substrate 103 and the first sensor substrate 101 are electrically connected to each other via the spring connector, but both substrates may be integrally formed. That is, the horizontal portion 103a of the bezel conductive substrate 103 may also serve as the first sensor substrate 101.

### REFERENCE SIGNS LIST

1 . . . Biological information measurement device
10 . . . Main body portion
11 . . . Main body case
12 . . . Display
13a, 13b . . . Operation button
14 . . . Lug
15 . . . Sensor substrate housing portion
16 . . . Cuff cover
17 . . . Control board
20 . . . Belt portion
21 . . . Belt
22 . . . First pressing cuff
23 . . . Second pressing cuff
24 . . . Sensing cuff
25 . . . Surface fastener
50 . . . Electrocardiographic signal detection circuit
100 . . . Sensor substrate set
101 . . . First sensor substrate
102 . . . Second sensor substrate
103 . . . Bezel conductive substrate
106 . . . Screw member
111 . . . First LED
112 . . . First PD
113 . . . Second LED
121 . . . Second PD
131 . . . Bezel (first electrode)
132 . . . Second electrode
133 . . . Third electrode
151 . . . Resin cover
152 . . . Isolation wall
161 . . . Piezoelectric pump
162 . . . Valve
163 . . . Pressure sensor
164 . . . Flow path plate
165 . . . First connection portion
166 . . . Second connection portion
182, 183, 184 . . . Spring connector
191 . . . Rechargeable battery
D1, D2 . . . TVS diode
TP . . . Conductive tape
TS . . . Contact surface

1. A biological information measurement device used by being attached to a human body, including at least a first electrode and a second electrode, and configured to measure an electrocardiographic waveform based on a potential difference between the first electrode and the second electrode, the biological information measurement device comprising:
a main body case including a side surface formed of a conductive material and configured to function as the first electrode, a bottom surface on which the second electrode is disposed and which abuts against the human body when being attached, and a top surface facing the bottom surface;
an electrocardiographic signal detection circuit configured to detect signals related to potentials of the first electrode and the second electrode;
a first electrical path configured to electrically connect the first electrode to the electrocardiographic signal detection circuit; and
a second electrical path on which an electrostatic discharge protection element is mounted and configured to connect the first electrode to the second electrode via the electrostatic discharge protection element.

2. The biological information measurement device according to claim 1, wherein
the electrocardiographic signal detection circuit is provided on a first substrate disposed in the main body case in a direction parallel to the bottom surface, and
a part of the first electrical path and a part of the second electrical path are provided on a second substrate that is disposed in the main body case in a direction orthogonal to the bottom surface and is joined to the first electrode by a conductive induction tape to be electrically connected to the first electrode.

3. The biological information measurement device according to claim 2, wherein
the first substrate and the second substrate are integrally formed as a rigid flexible substrate.

4. The biological information measurement device according to claim 1, wherein
a display is provided on the top surface, and
the first electrode and the display are configured to be electrically connected to a conductive tape.

5. The biological information measurement device according to claim 2, further comprising:
a third substrate disposed in a direction parallel to the first substrate in the main body case and on which a control device configured to control the biological information measurement device is mounted; and
a fourth substrate provided with an operation button disposed to protrude from the side surface and a part of an electrocardiographic signal line configured to connect the electrocardiographic signal detection circuit to the control device, and disposed in a direction orthogonal to the first substrate at a position different from the second substrate.
